(19) United States
(12) Patent Application Publication
(10) Pub. No.: US 2009/0312639 A1
(43) Pub. Date: Dec. 17, 2009

(54) ACOUSTIC IMAGING METHOD AND APPARATUS

(76) Inventors: Andrew John Medlin, South Australia (AU); Andrew John Paul Niemiec, South Australia (AU); Stewart Gavin Bartlett, South Australia (AU); Roger Michael Costello, Western Australia (AU)

Correspondence Address: Intellectual Property Dept.;Dewitt Ross & Stevens SC, 2 East Mifflin Street, Suite 600, Madison, WI, 53703-2865, US

(21) Appl. No.: 12/307,305

(22) PCT Filed: Jul. 2, 2007
(86) PCT No.: PCT/AU07/00915

§ 371 Date: Jan. 2, 2009

(30) Foreign Application Priority Data
Jul. 7, 2006 (AU) 2006904357

(51) Int. Cl. A61B 8/14

(52) U.S. Cl. 600/443

(57) ABSTRACT

A method of and apparatus for ultrasound imaging whereby an acoustic transmit signal including at least one transmit acoustic frequency is transmitted such that at least some of the acoustic energy of the pulse is transmitted into a body to be imaged being of material which has a response to the acoustic energy which will produce a demodulation of the transmit pulse to produce a demodulated signal; and an acoustic receive comprising echoes of the demodulated signal is received at a frequency that is approximately equal to the frequency of the demodulated signal.

### FIELD OF THE INVENTION

The present invention relates to a method and apparatus for the transmission and reception of acoustic waves for the purpose of imaging.

### BACKGROUND OF THE INVENTION

A variety of equipment and methods exist for imaging using ultrasound energy, with applications in medical imaging, industrial non-destructive testing, and underwater imaging. In general, these Systems transmit an ultrasound pulse and wait for returned echoes generated by changes of impedance of the structures being imaged. The returned echoes are processed and displayed on a screen as an image, a graph, or some other format. The quality of the image and data generated is dependent on many factors, but two important factors are the beam width at the point of reflection and the length of the pulse transmitted. Narrow beams provide improved lateral resolution and short pulses provide improved axial resolution.

The early ultrasound systems used a single ceramic transducer operating in A-mode, or amplitude mode. These systems transmitted an ultrasound pulse and waited for the received echoes, and plotted the amplitude of the received echoes versus time. Advances of this system included static B-mode systems, where the transducer was mounted on a mechanical arm, and the returned echoes were used to draw a grey scale image. These systems were still in use up to the mid 1980s, but were then rapidly replaced with real-time beam-forming systems.

Early beam-forming transducers were experimented with in the early 1970s. By the mid 1980s the construction and operation had improved enough that the image quality started to surpass the static B-mode systems. Beam-forming transducers have a large number of ceramic elements manufactured in an array, and include linear transducers, phased-array transducers, and convex transducers. The most common transducers are 1D arrays where the beams of the transducers can be generated in different directions or offsets in the same plane to create an image. The number of ceramic elements is usually between 64 and 256 elements, and improvements in methods have included transmit focusing, where users can select the region of transmit focus; and receive beam-forming, where a greater gain is applied to particular scan lines relative to the gain applied to other scan lines. The improvements are all relevant in the scan plane, but in the plane perpendicular to the scan plane a fixed focal length is used and beam width and beam divergence are important considerations.

Methods to improve focus in both the scan plane and the perpendicular plane exist. The most common is the construction of 1.5D arrays, where the scan plane contains a row of elements (usually greater than 64 to 266) and the perpendicular plane contains a small column of elements (usually 4). The resultant array enables dynamic focus in both the scan plane and the perpendicular plane, but the large number of elements results in a very expensive system.

The quality of any ultrasound imaging system is affected by the side-lobes produced by the transducer. All transducers produce side-lobes when transmitting a pulse. Side lobes reduce the effective efficiency of the transducer, but more importantly are a source of noise in the receive signal. The relative intensity of the side-lobes is reduced by using higher frequency transducers, therefore a transducer system using higher frequency pulses will have superior transmit characteristics to a transducer system using lower frequency pulses, at least in part due to the reduction in side lobes.

Axial resolution (also known as the depth, linear, longitudinal and range resolution) is the minimum distance in the beam direction between two reflectors which can be identified as separate echoes. The axial resolution is slightly more than half the spatial pulse length, which is the number of waves in the transmitted ultrasound pulse multiplied by their wavelength.

Transducer bandwidth and pulse length are related. Theoretically, only infinite sine waves have a single frequency. The beginning and end of an ultrasound pulse introduce a range of frequencies; the shorter the pulse, the wider its frequency spectrum. A low bandwidth transducer will respond to a short voltage pulse with a relatively long lasting vibration, emitting ultrasound with a narrow bandwidth, but a long pulse length. This gives poor axial resolution.

A broadband transducer will emit a short pulse of ultrasound consisting of a broad range of frequencies, which will improve axial resolution, but there are limitations to the width of passband which can be achieved with practical transducers. There is also the problem that increasing transducer bandwidth leads to reduced efficiency in driving the transducer.

There exists a need to obtain improvements in ultrasound imaging technology to enable improved image quality without increased cost.

### SUMMARY OF THE INVENTION

In one form the invention may be said to lie in a method of ultrasound imaging including the steps of transmitting a transmit signal including at least one transmit acoustic frequency such that at least some of the acoustic energy of the pulse is transmitted into a body to be imaged, said body being of material which has a response to the acoustic energy which will produce a demodulation of the transmit pulse to produce a demodulated signal;

receiving an image signal at a receive frequency,
wherein said receive frequency is approximately equal to the frequency of the demodulated signal, the image signal substantially comprising echoes of the demodulated signal.

In preference, the transmit signal is a discrete transmit pulse and the demodulated signal is correspondingly a demodulated pulse, the length of the transmit pulse being controlled such that the demodulated pulse is no more than two cycles in duration.

In preference, the length of the transmit pulse is controlled such that the demodulated pulse is no more than one cycle in duration.

In a further form of the invention the transmit signal includes a second transmit acoustic frequency and the receive frequency is approximately a difference frequency said difference frequency being determined as the frequency difference between said first frequency and said second frequency.

In a further form the invention may be said to reside in an apparatus for ultrasound imaging including a first transducer adapted to transmit an acoustic transmit signal in response to an applied electrical excitation signal and to produce an electrical received signal in response to an acoustic receive signal, electrical transmit circuitry adapted to produce a user variable electrical excitation signal, electrical receive circuitry adapted to process the electrical received signal, a display device adapted to display the results of said processing, a body to be imaged being of material which has a response to the acoustic energy which will produce a demodulation of the transmit pulse to produce a demodulated signal; wherein the receive signal substantially comprises echoes of the demodulated signal the transmit signal being a discrete transmit pulse and the demodulated signal being correspondingly a demodulated pulse, the length of the transmit pulse being controlled such that the demodulated pulse is no more than two cycles in duration.

In preference, the apparatus is further adapted to control the length of the transmit pulse such that the demodulated pulse is no more than one cycle in duration.

### BRIEF DESCRIPTION OF THE DRAWINGS

FIG. 1 shows a schematic representation of a hand held ultrasound apparatus incorporating an embodiment of the invention.

FIG. 2 shows a plot of a frequency spectrum of a transmit transducer.

FIG. 3 shows an idealised excitation pulse in the time and frequency domains.

FIG. 4 shows a transmit signal in accordance with the invention, in the time and frequency domains.

FIG. 5 shows the transmit signal of FIG. 4 as it would be at 60 mm depth in human tissue, in both the time and frequency domains.

FIG. 6 shows a transmit signal of the prior art.

FIGS. 7a-f. show simulations of an ultrasound signal of the prior art at succeeding penetration depths.

FIGS. 8a-f. show simulations of an ultrasound signal in accordance with the invention at succeeding penetration depths.

FIG. 9 shows resolution results of a simulation of a prior art system.

FIG. 10 shows resolution results of a simulation of a system utilising the invention.

FIG. 11 shows a simulation of a waveform and corresponding frequency spectrum a Gaussian enveloped sine wave for a transmit signal with a peak at a frequency of 10.5 MHz.

FIG. 12 shows the demodulated signal for the system of FIG. 11.

FIG. 13 shows a simulated long burst transmit signal in the time and frequency domain.

FIG. 14 shows the demodulated signal for the system of FIG. 13.

FIG. 15 shows a transmit signal for an alternative embodiment of the invention.

FIG. 16 shows the demodulated signal for the system of FIG. 15.

### DETAILED DESCRIPTION

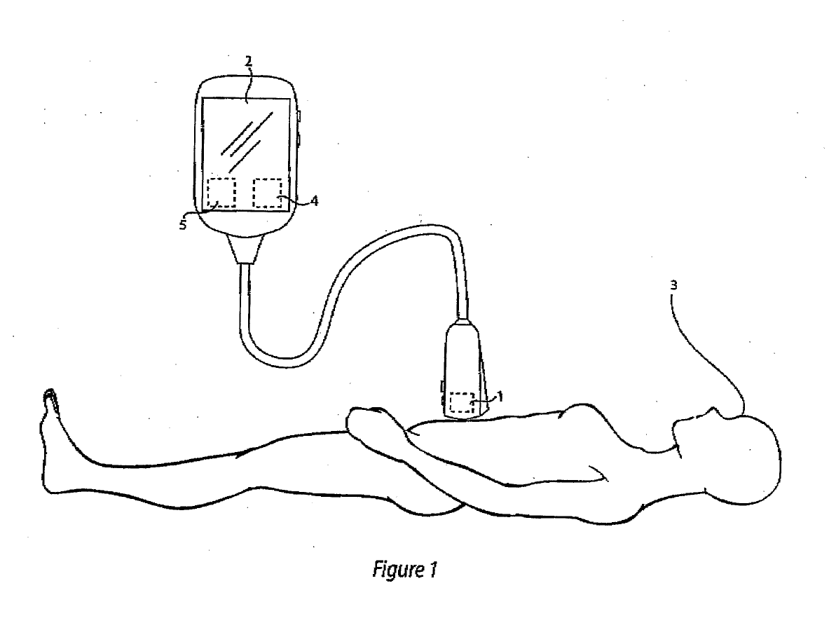

FIG. 1 shows a handheld ultrasound transmission, reception and analysis device, schematically represented in use in a medical diagnostic setting. The illustration is not to scale.

An acoustic transmit signal is transmitted into a medium to be imaged 3 by use of a broadband piezoelectric transducer 1.

A receive signal is generated by the interaction of the transmit signal with the medium to be imaged, the target medium.

The receive signal is received by transducer 1, and the resulting receive signal is analysed by receive circuitry.

The results of the analysis are displayed on an image forming display 2 and are referred to as an image, but it should be understood that image forming is not an essential part of the method and the results of the analysis of the received signal may be communicated to a user or other recipient in any appropriate way, including, but not limited to: audible sounds, text displays and lights or patterns of lights.

The transducer 1 acts as a passband filter, with the pass frequency being the resonant frequency of the transducer crystal. The transducer is stimulated to oscillate by means of an electrical excitation signal tuned to a desired excitation frequency, provided by excitation control circuitry 4. This excitation frequency lies within the passband of the transducer. The filtering of the electrical signal by the transducer generates the waveform of the acoustic transmit signal.

The invention may be embodied in a hand held medical diagnostic device as shown in FIG. 1, or in any other configuration in which ultrasound equipment is made or used.

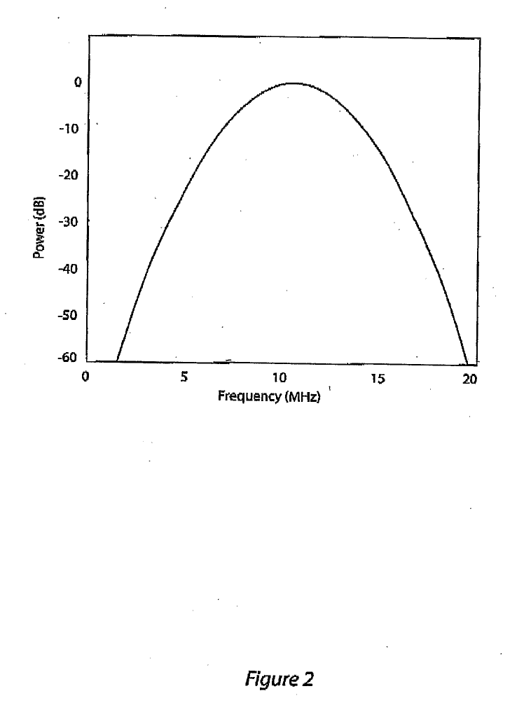

The frequency spectrum of the transducer 1 is in the form of a broad pass band with a peak centred on a desired carrier frequency, which typically would be a frequency in the range 8-16 MHz. However, carrier frequencies outside this range may also be used. In the embodiment illustrated, the transducer 1 has a transducer frequency spectrum with a peak at 10.5 MHz as shown in FIG. 2.

Preferably, there is transmitted a single transmit carrier frequency within a pulse of short duration. The pulse duration and the spectral content of the signal are related such that the shorter the pulse duration, the broader the spectral content of the signal. In order to pass all the spectral content of the excitation signal and so preserve the short pulse length, the transducer must have a sufficiently broad pass band.

The larger the bandwidth, the shorter the acoustic output signal in the time domain, but the lower the conversion efficiency from electrical to acoustic energy. The spectrum of FIG. 2 has a bandwidth which yields about 70% efficiency.

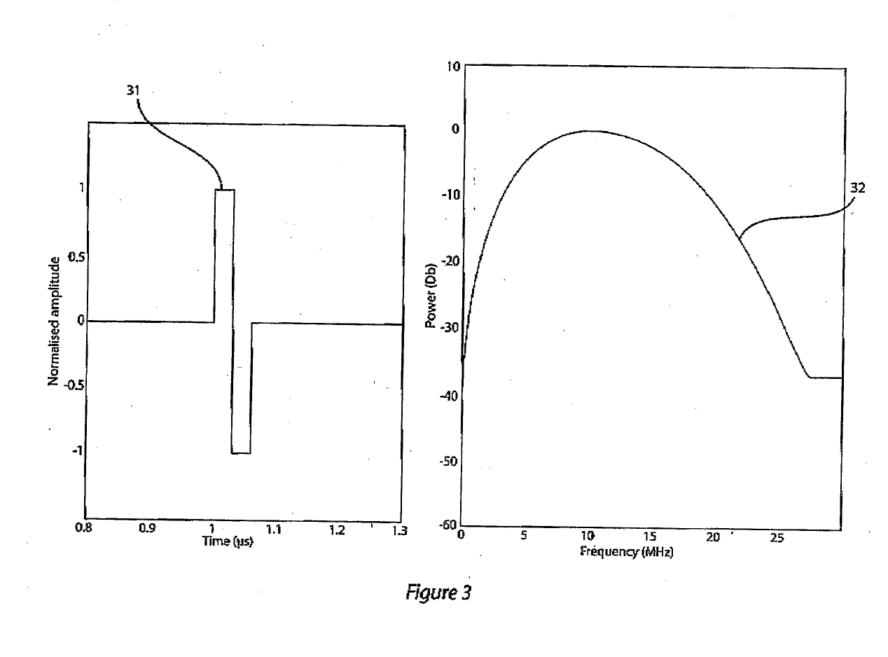

The transmitting transducer is preferably broadband by design so that it can pass the broad range of frequencies required to generate a transmit signal of short pulse duration. A transmit signal is generated by stimulating a transducer 1 with a cyclic electronically controlled signal. In a preferred embodiment, this is a pulse, called an excitation pulse. An exemplary excitation pulse 31 is shown in FIG. 3. The excitation pulse is spectrally filtered by the transducer to produce a transmit signal pulse.

The electronic excitation pulse excites vibrations in the transducer which propagate into the surrounding medium in the form a directed beam of acoustic waves. The excitation pulse itself consists of a spectrum of frequencies. The short pulsed nature of the excitation means that the spectral content of the excitation pulse will also be broadband.

Preferably, the excitation pulse is generated in such a way that the peak in its frequency spectrum coincides with the peak pass band of the transducer, in this way ensuring the optimal conversion of electrical into acoustic energy as the transmit transducer filters the excitation signal.

The excitation pulse amplitude is typically in the order of ±100 Volts, although this may vary widely, depending on the application. In order to maximise the signal-to-noise ratio for imaging, the excitation signal is preferably maximal to maximise the strength of the transmit signal within component and safety constraints.

The transmit signal from the transducer results from the conversion of electrical energy in the form of an excitation pulse into acoustic energy. The spectral content of the acoustic transmit pulse consists of the spectral content of the excitation pulse 31 filtered by the spectral response of the transmit transducer 1.

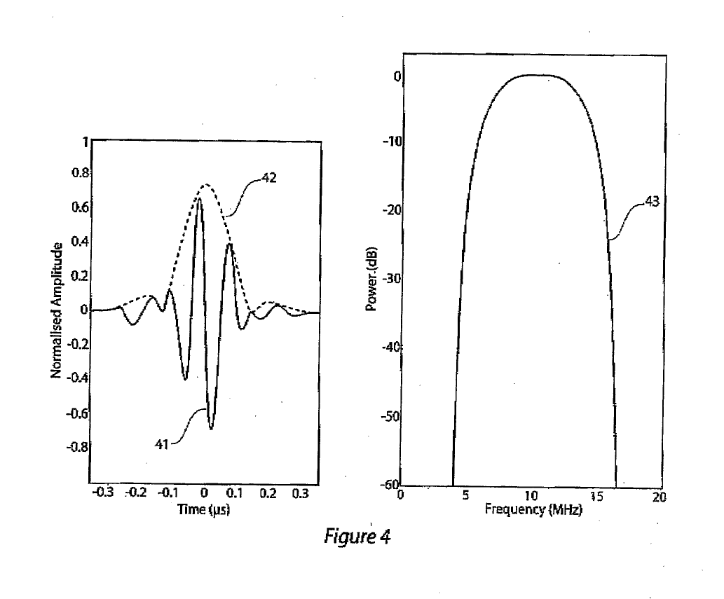

Applying the electrical excitation signal shown in FIG. 3 to the transmit transducer 1 having the frequency response as illustrated in FIG. 2 results in a transmit signal 41 centred on 10.5 MHz as shown in FIG. 4.

The shape of the transmit signal pulse 41 can be traced by its envelope 42, as shown in FIG. 4. For a transducer of 70% bandwidth, the transmit signal contains approximately 3 cycles of the carrier frequency. The envelope pulse length is the envelope width centred on the peak. It is the envelope pulse length of the demodulation signal which finally determines the imaging resolution in the direction of wave propagation.

As large a wave amplitude as possible is desirable, within practical and safety limits, in order to maximise the strength of the demodulated receive signal.

The acoustic transmit signal from the transmit transducer propagates into the target medium in a directed beam. For media with nonlinear properties (such as human tissue), the signal demodulates to a short, low frequency pulse.

The demodulated signal is a highly penetrating signal with the resolution typically associated with a much higher frequency. This demodulated signal is reflected by features in the media to be imaged and forms the receive signal.

The receive signal is received by transducer 1 and the resultant electronic signal is transmitted to receive electronics 5, for analysis and display on the image forming display 2.

In other embodiments, the receiving transducer may be a separate transducer to the transmitting transducer. In either case, the receive transducer constitutes an additional filter to the acoustic signal prior to reception by the receive electronics.

The demodulation frequency can be arbitrarily tuned to within a factor of N of the carrier frequency, where N is the approximate number of cycles in the carrier pulse (for a 70% bandwidth, N˜3).

Even though a single cycle of a square wave is applied to the transducer, multiple cycles of acoustic output are produced. The broader the bandwidth of the transmit transducer, the fewer the cycles in the transmit signal, but with a corresponding loss of efficiency (loss of acoustic pulse amplitude).

The demodulation centre frequency is related to the width of the envelope of the carrier pulse. The shorter the envelope, the higher the centre frequency of the demodulated signal. This permits arbitrary tuning of the demodulation frequency to any frequency within a factor of N of the carrier frequency. To a good approximation, the fully demodulated signal in the time domain tends towards the shape of the second derivative (i.e. the curvature) of the envelope squared of the initial transmit signal.

For highly attenuating nonlinear media with properties comparable to human tissue, and with an initial acoustic signal of high carrier frequency (8-16 MHz), complete demodulation occurs over a short distance (typically a few cm). After this distance, the demodulated waveform continues to propagate through the medium with much lower attenuation than the original transmit signal because the signal is predominantly low frequency. As discussed, signal attenuation in the target media increases with increasing signal frequency.

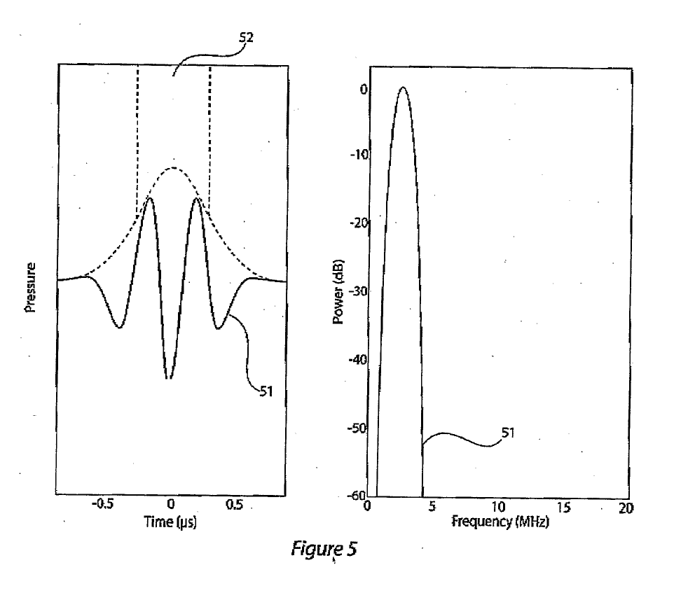

This is shown in FIG. 5, showing the transmit signal of FIG. 4 as it would be at 60 mm depth in human tissue, in both the time and frequency domain. As can be seen, the attenuation of the signal at the transmitted frequency of 10.5 MHz is essentially complete. No useful imaging at this depth could be achieved by receiving a signal at the transmit frequency.

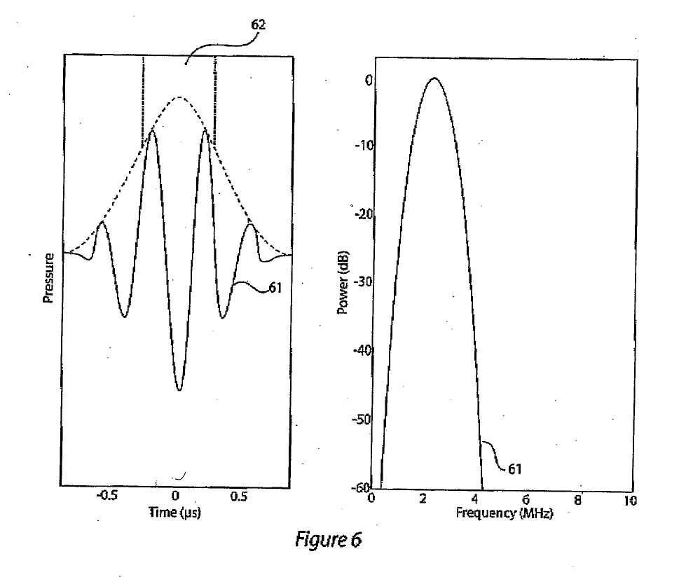

The conventional solution to this would be to use a lower transmit frequency to achieve greater penetration. This would give the result illustrated in FIG. 6. This shows a transmit signal of 3.5 Mhz, also as it would be at 60 mm depth in human tissue. As can be seen, significant energy remains at the transmit frequency of 3.5 MHz, and useful imaging can be done by receiving at the transmit frequency. The receive signal 61 is shown.

An advantage of the present invention may be seen when comparing the pulse length of the receive signals. The receive signal in the case of FIG. 5 is signal 51, also at 3.5 MHz. The effective pulse length 52, is about 1.01 mm. The effective receive signal pulse length 62, for the direct transmission case of FIG. 6 is 1.23 mm.

Axial resolution is a function of receive signal pulse length. The pulse length for the demodulated signal of the invention is significantly shorter than that of the prior art method of transmitting directly at the desired receive signal frequency. This leads to improved axial imaging resolution.

A further advantage of the present invention stems from the fact that the amplitude of the demodulation signal increases as the frequency decreases. In order to generate a demodulated signal, the pressure wave needs to be of sufficient amplitude, as the nonlinear effect is proportional to the pressure. At lower frequencies, the attenuation is correspondingly lower, resulting in an increased transfer of energy to the demodulation frequency at greater depth. This is equivalently stated by the relationship of the Gol'dberg number to the carrier frequency:

$$Γ = \frac{β {p}_{0}}{π D {ρ}_{0} {f}_{c}}$$

where β=nonlinearity parameter, p₀=source pressure, D=sound diffusivity (proportional to the attenuation coefficient), ρ₀=ambient density and f_{c}=carrier frequency. As the carrier frequency increases, the Gol'dberg number decreases, meaning that the nonlinear mechanism has a relatively weaker effect.

The energy available at the imaging frequency at the imaging depth is obviously a prime determinant of image quality, since it determines the maximum strength of the receive signal.

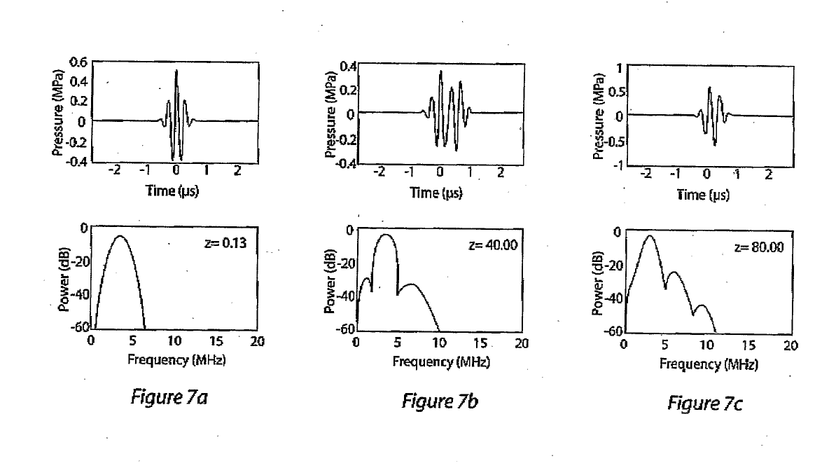

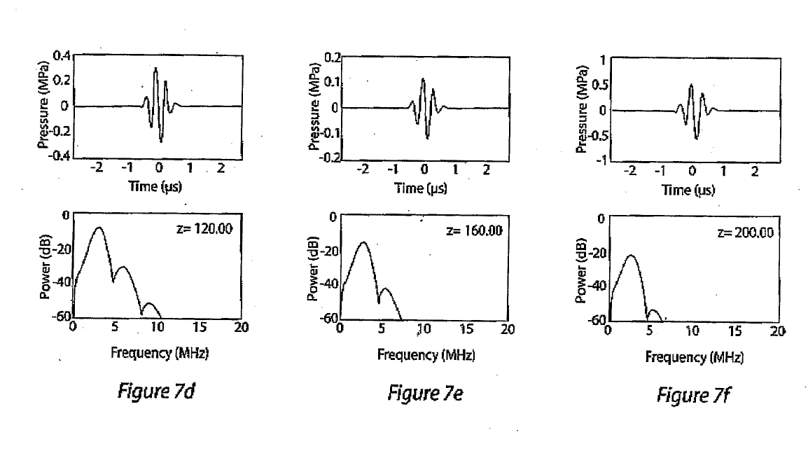

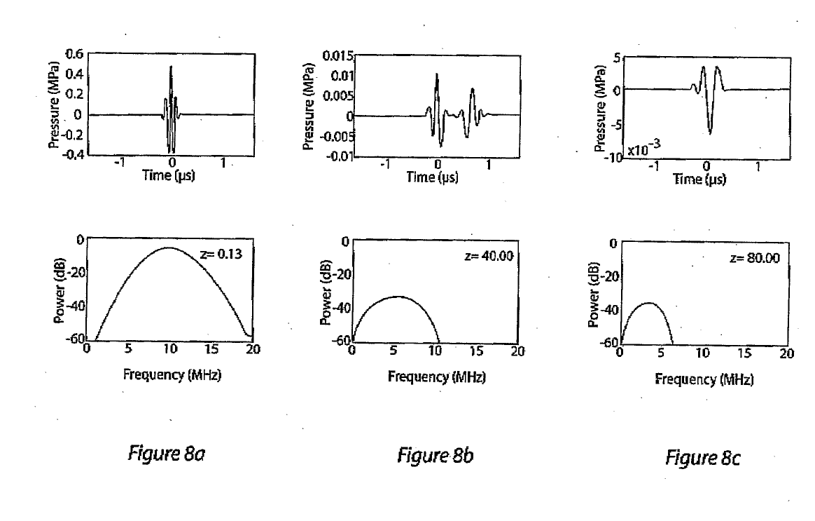

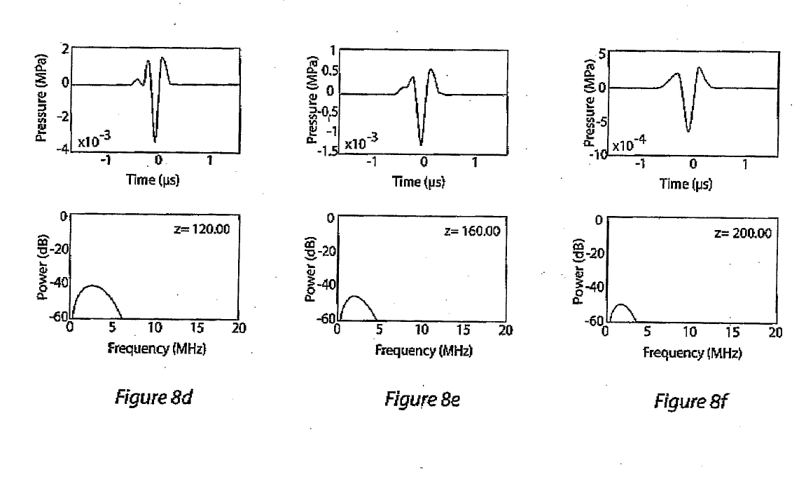

This is illustrated in FIGS. 7 and 8. These show simulations of an ultrasound signal from a transducer with a radius of 10 mm and an acoustic focal length of 125 mm. Each figure shows a transmit signal in both the time and frequency domains in human tissue at 6 depths: 013 mm, 40 mm, 80 mm, 120 mm, 160 mm and 200 mm.

FIG. 7 illustrates the prior art, where a lower frequency transmit signal (in this case 3.5 MHz) is chosen to give useful acoustic energy penetration for imaging, with the receive signal being received at the same frequency as the transmit frequency.

The computed time and frequency domain signals are shown in FIG. 7a-f. FIG. 7a shows the initial waveform and frequency spectrum.

A simulation of a 10.5 MHz transmit signal demodulating to a 3.5 MHz receive signal, in accordance with the invention, is shown in FIG. 8. The computed time and frequency domain signals are shown in FIG. 8a-f. FIG. 8a shows the initial waveform and frequency spectrum.

The results of FIG. 8 show that for a 10.5 MHz carrier signal in human tissue, demodulation is complete by about 80 mm depth. From this depth onwards, we see a very short, low frequency pulse containing about 2 cycles of the demodulation frequency of 3.5 Mhz. Such a short pulse cannot be produced by the prior art method of direct electronic excitation of a 3.6 MHz transducer.

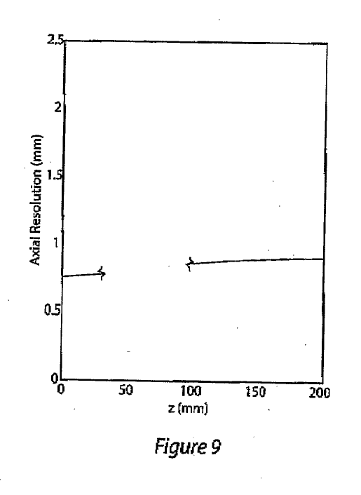

The axial and lateral resolution obtained for the 3.5 MHz direct transmission prior art case are shown in FIG. 9. FIG. 9 shows a minimum beam width of about 3.4 mm at a depth of 80 mm, at which point the axial resolution is about 0.87 mm. It can be seen that the axial resolution remains almost constant to 200 mm depth.

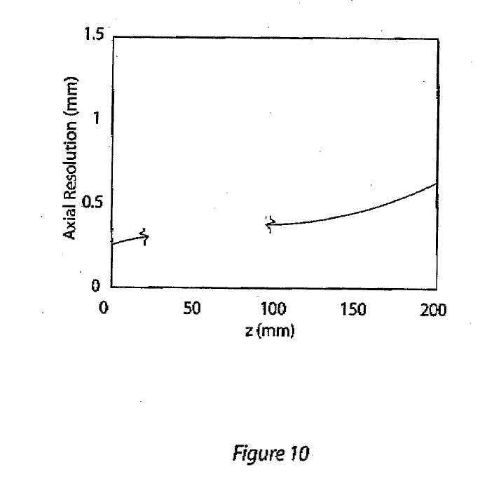

For the example of the current invention, as illustrated in FIG. 8, the axial and lateral resolution is shown in FIG. 10. The minimum beam width is 3.3 mm at about 95 mm depth, with a corresponding axial resolution of 0.4 mm. This is a significant improvement over the prior art case, and allows for features to be resolved which are have less than half the separation that would be required in the prior art case.

It can be seen from FIG. 10 that even at the maximum illustrated 200 mm depth, the axial resolution is 0.63 mm, which is still better than the best axial resolution for the prior art case, illustrated in FIG. 9, of 0.87 mm axial resolution.

In a further embodiment, the demodulation process is employed to generate a single cycle, low frequency pulse which approaches the shortest practically possible duration. This allows axial resolution very close to the absolute theoretical maximum to be achieved.

As stated previously, the demodulation pulse waveform, when fully developed and neglecting attenuation, assumes the shape of the second derivative squared of the envelope of the transmit pulse. Geometrically, the second derivative is interpreted as the curvature of the envelope shape. Positive curvature is called concave (opens upwards), and negative curvature is called convex (opens downwards).

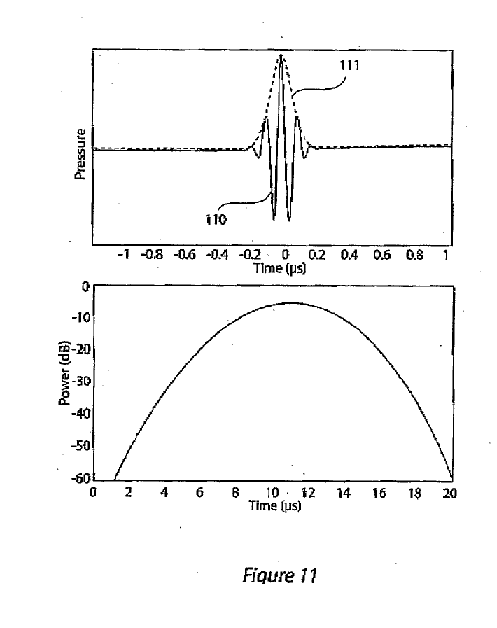

FIG. 11 shows a simulation of a waveform and corresponding frequency spectrum of a Gaussian enveloped sine wave for a transmit signal with a peak at a frequency of 10.5 MHz.

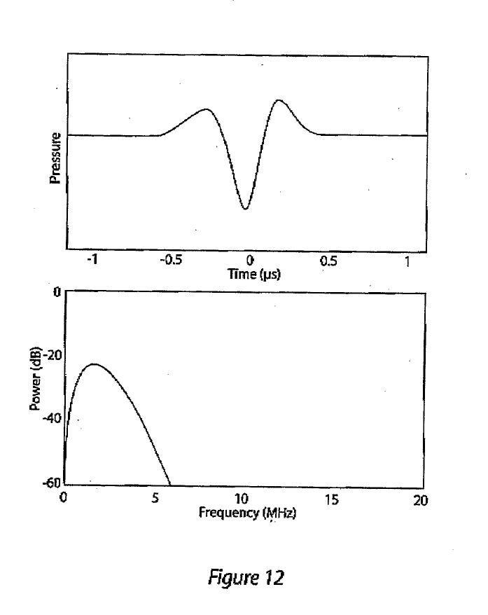

At the beginning of the Gaussian envelope, the curvature is positive, in the middle of the envelope about the peak it is negative, and at the end it is positive again. This gives rise to the demodulated signal shown in FIG. 12. The degree of curvature, which is greatest in the negative curvature region about the peak of the Gaussian envelope, corresponds directly to the magnitude of the demodulated signal where the negative peak is of higher magnitude than the positive peaks.

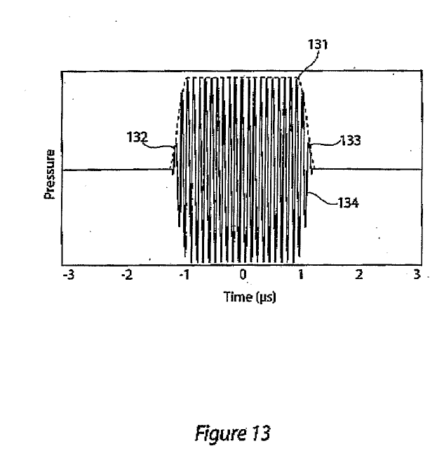

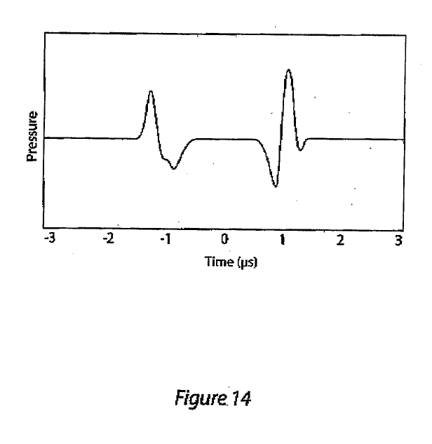

A simulated long burst of tone is illustrated in the time and frequency domain in FIG. 13. The demodulated signal is shown in FIG. 14.

During a long tone burst, the envelope has high curvature at the beginning and end of the pulse, but in the middle the curvature is zero. As illustrated in FIG. 14, the demodulated signal consists of a separated pair of short, quasi-single cycle pulses, one corresponding to the initial rise of the envelope and the other corresponding to the fall of the envelope.

Each pulse in the pair resembles a quasi ideal, shortest possible acoustic pulse. Comparison of the demodulation signal in FIG. 14 with that of FIG. 5 leads to the conclusion that the demodulated signal of FIG. 5 may be seen as a merged pair of such pulses from the rise and fall stages of the Gaussian envelope.

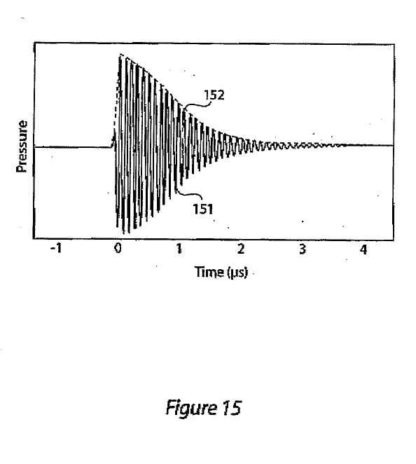

In this further embodiment, a transmit signal in the form of a slow decay signal burst is transmitted by transducer 1. The transmit signal is illustrated in FIG. 15. As can be seen, the rise time of the envelope of the signal is short, and the decay time is relatively long.

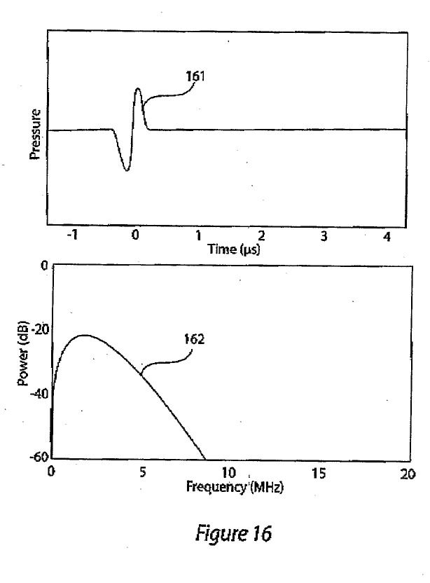

The demodulation signal, as shown in FIG. 16 includes a high amplitude, quasi-single cycle pulse derived from the envelope rise, and a much weaker amplitude pulse derived from the fall.

The shape of the fall in the envelope may be controlled such that its curvature is sufficiently small such that the demodulated waveform is very close to a low frequency, single cycle pulse. Such a pulse is close to the ideal, theoretically best possible waveform for imaging. It is not possible to generate such an ideal waveform directly with a finite bandwidth transducer.

With such a single cycle, low frequency pulse, the highest possible axial resolution is achieved because the pulse is as short as possible. Simulation shows that such a demodulated waveform can be generated in the first few cm of tissue, making it feasible for imaging at low frequency and high resolution from a few cm onwards.

It is advantageous to produce a continuous transmit signal for imaging. This is particularly the case for Doppler imaging. A continuous, single frequency signal will not produce a demodulation effect. The envelope of such a signal is a straight line, and, in the theoretical case of a perfect single frequency signal of unvarying amplitude, no demodulation signal is produced.

In a further embodiment, a transmit signal combines pulses at each of two frequency components, f₁ and f₂. This is transmitted by a transmit transducer into a medium to be imaged having non-linear acoustic response, such as human tissue. The demodulation signal produced by the interaction between the two components in the non-linear medium will be a pulse signal at the beat or difference frequency, f₁-f₂. A receive signal for imaging is received at this demodulation frequency by a receive transducer, which may be the same transducer as the transmit transducer.

As in the illustrated embodiment, the pulse length of the transmit pulse in a practical system will be several wavelengths. The demodulation signal, at a lower frequency, will be of a lesser number of wavelengths. This will give an improvement in the axial resolution of the imaging.

The transmit pulse length and the transmit pulse frequencies may be chosen to reduce the pulse length of the demodulation frequency to a single wavelength, for the greatest improvement in axial resolution.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1-18. (canceled)

18. A method of ultrasound imaging comprising the steps of:
a. transmitting an acoustic transmit signal including at least one transmit acoustic frequency into a body to be imaged, the body being of material which has a response to the acoustic transmit signal which will produce a demodulation of the transmit signal to produce a demodulated signal having a frequency lower than the transmit signal frequency;
b. receiving an acoustic receive signal at a receive frequency, wherein:
(1) the receive frequency is approximately equal to the frequency of the demodulated signal, and
(2) the receive signal substantially comprises echoes of the demodulated signal.

19. The method of claim 18 wherein:
a. the transmit signal is a discrete transmit pulse and the demodulated signal is correspondingly a demodulated pulse,
b. the length of the transmit pulse:
(1) is greater than two wavelengths of a signal at the transmit acoustic frequency,
(2) is controlled such that the demodulated pulse is no more than two cycles in duration.

20. The method of claim 19 wherein the length of the transmit pulse is controlled such that the demodulated pulse is no more than one cycle in duration.

21. The method of claim 18 wherein the acoustic transmit signal consists of a pulse of multiple wavelengths duration at the transmit acoustic frequency, the pulse having a smoothly varying amplitude wherein the amplitude rise time is significantly less that the amplitude decay time, such that the demodulated signal is no more than two cycles in duration.

22. The method of claim 21 wherein the demodulated signal is no more than one cycle in duration.

23. The method of claim 18 wherein:
a. the transmit signal includes a second transmit acoustic frequency, and
b. the receive frequency is a difference frequency, the difference frequency being determined as approximately the frequency difference between the first frequency and the second frequency.

24. The method of claim 23 wherein the length of the transmit pulse is controlled such that the demodulated pulse is no more than one cycle in duration.

25. The method of claim 23 wherein the demodulated signal is no more than one cycle in duration.

26. An apparatus for ultrasound imaging of a body comprising:
a. a first transducer which transmits an acoustic transmit signal in response to an applied user variable electrical excitation signal and produces an electrical receive signal in response to an acoustic receive signal,
b. electrical transmit circuitry which produces the user variable electrical excitation signal,
c. electrical receive circuitry which processes the electrical receive signal,
d. a display device which displays the results of the processing,
the body to be imaged being of material which has a response to the acoustic transmit signal which will demodulate the transmit signal to produce a demodulated signal,
the transmit signal being a discrete pulse and the demodulated signal being correspondingly a demodulated pulse, and
wherein the acoustic receive signal substantially comprises echoes of the demodulated signal.

27. The apparatus of claim 26 wherein the electrical excitation signal is controlled by the electrical transmit circuitry to be of a selected length to ensure that the acoustic transmit signal pulse is of a length such that the demodulated pulse is no more than two cycles in duration.

28. The apparatus of claim 27 wherein the electrical transmit circuitry controls the electrical excitation signal such that the length of the acoustic transmit signal pulse such that the demodulated pulse is no more than one cycle in duration.

29. The apparatus of claim 26:
a. wherein the first transducer transmits the acoustic transmit signal in response to an applied electrical excitation signal, and
b. further including a second transducer which produces the electrical receive signal in response to the acoustic receive signal.

30. The apparatus of claim 29 wherein the second transducer is a transducer array.

31. The apparatus of claim 26 wherein the acoustic transmit signal consists of a pulse of multiple wavelengths duration at the transmit acoustic frequency, the pulse having a smoothly varying amplitude wherein the amplitude rise time is significantly less that the amplitude decay time, such that the demodulated signal is no more than two cycles in duration.

32. The apparatus of claim 31 wherein the demodulated signal is no more than one cycle in duration.

33. The apparatus of claim 26 wherein the ultrasound apparatus is a hand held diagnostic ultrasound unit.

34. The apparatus of claim 26 wherein the first transducer is a single element ultrasound transducer.

35. The apparatus of claim 26 wherein the first transducer is a transducer array.

36. A method for producing an ultrasound image having improved axial resolution comprising:
a. selecting a desired imaging frequency appropriate for the depth at which imaging is to take place,
b. calculating a frequency and a pulse length for an acoustic transmit signal pulse which will demodulate within a body to be imaged to the imaging frequency;
c. transmitting a transmit pulse of the calculated frequency and length into the body to be imaged,
d. receiving an ultrasound imaging acoustic signal at the imaging frequency.

37. The method of claim 36 wherein the ultrasound imaging acoustic signal is a discrete pulse with a duration of less than one cycle.
